(19) United States
(12) Patent Application Publication
(10) Pub. No.: US 2007/0166242 A1
(43) Pub. Date: Jul. 19, 2007

(54) ORAL CARE PRODUCT AND METHOD FOR THE REDUCTION OF DENTAL CARIES THROUGH INCLUSION OF MANNAN AND GALACTOMANNAN POLYSACCHARIDES IN DENTIFRICE FORMULATIONS

(76) Inventor: Robert D. Kross, Bellmore, NY (US)

Correspondence Address: EDWIN D. SCHINDLER, FIVE HIRSCH AVENUE, P.O. BOX 966, CORAM, NY, 11727-0966, US

(21) Appl. No.: 11/611,930

(22) Filed: Dec. 18, 2006

Related U.S. Application Data
(60) Provisional application No. 60/751,491, filed on Dec. 19, 2005.

(51) Int. Cl. A61K 9/68; A61K 8/73

(52) U.S. Cl. 424/48; 424/49

(57) ABSTRACT

An oral care composition for reducing colonization of the oral cavity by plaque and disease-causing microorganisms, includes saccharides containing cis-hydroxy sugar units or a derivative thereof. The oral care composition is a dentifrice, mouthwash, chewing gum or a lozenge. The saccharides include monomeric sugars with the cis-hydroxy stereoisomeric structure, polysaccharides, which include such sugars as food gums and their partially hydrolyzed reaction products. The cis-hydroxy saccharides are, for example, mannose, a mannose derivative, galactose, a galactose derivative, galactomannans, galactosarnine, fucose, arabinose, and rhamnose, and the food gums, and hydrolyzates thereof that include them are xanthan, konjac, guar gum, locust bean gum, tara bean gum and carrageenan.

### CROSS-REFERENCE TO PROVISIONAL PATENT APPLICATION

Domestic priority is hereby claimed, pursuant to 35 U.S.C. §119(e), from U.S. Patent Provisional Application No. 60/751,491, filed Dec. 19, 2005, the entire disclosures of which shall be deemed to be incorporated by reference herein.

### BACKGROUND OF THE INVENTION

1. Technical Field of the Invention

The present invention relates to an oral care product and related method for reducing the level of undesirable microorganisms in the oral cavity by including certain carbohydrate materials in products used in that area.

More particularly, the present invention relates to an oral care product and related method directed to saccharides containing sugar units of preferred spatial configuration, such as mannans and galactomannans, which structures promote adhesion to receptor sites on oral bacteria and thereby reduce their populations in the oral cavity. A preferred use of these materials is in dentifrices.

2. Description of the Prior Art

It is well known that many bacteria causing infections in humans, and other mammalian and non-mammalian species, have an affinity to bind to a certain sugar, mannose, as a component of the cell membranes of tissues. The inventor has taken advantage of this awareness, and the similar action of related sugar stereoisomers, both as monosaccharides and polysaccharides thereof, to teach the use of certain of these materials to suppress the attachment of bacterial pathogens to intestinal lumina of poultry and mammalian species, and thereby reduce the numbers of pathogens that would otherwise reside in such intestinal tracts (see, e.g., R. Kross, U.S. Pat. No. 6,126,961, issued Oct. 3, 2000, entitled “Composition and method for reducing the colonization of animal intestines by salmonella and other bacterial pathogens.”)

D-mannose is a monosaccharide sugar found naturally in fruits and is, for example, an epimer of glucose, having the same atomic composition except differing in their three-dimensional form. There are significant studies that indicate its involvement in immunological reactions, as well as reports on its therapeutic effect in urinary tract infections. For example cranberry juice, which contains much D-mannose, has a beneficial action in such infections. It has also been theorized that, in a manner similar to that taught in my above-referenced U.S. Patent, in intestinal infections, the D-mannose in fruit competes with receptor sites on the cells in the gut that bind infectious bacteria, causing them to leave their binding sites on the gut, and thereby have a beneficial effect in lessening the infection. This is consistent with the generally accepted belief that the majority of infectious diseases are initiated by adhesion of pathogenic organisms to the tissues of the host. The overwhelming majority of bacteria have what are called receptor sites or lectins that they use to hold on to cells in our bodies. These lectins can be analogized to hands that fit specific sugar complexes on the surface of cells and this is how they “hang on to them.” This attachment is the first step in starting an infection—if the bacteria are not attached they are washed out and cause no problems. Soluble carbohydrates, such as mannose, are recognized by the bacterial lectins and block the adhesion of the bacteria to the body tissues.

It is now recognized that a similar binding phenomenon applies to tooth surfaces. Teeth, when freshly cleaned by a so-called “prophy” procedure, rapidly acquire a protective, proteinaceous pellicle, through salivary action. Certain sugars, such as xylose and its alcohol, xylitol, are incorporated into misleadingly-termed “sugarless gums,” because of their proven ability to inhibit bacterial development on the surface of teeth. (Such gums are called sugarless because they have neither sucrose nor glucose, which are the sugars associated with cavities.) It is presumed that the xylose binds to receptor sites on the proteinaceous pellicle, and competes with the bacterial lectins (such as found in the cavity-forming bacteria Strep. mutans and Strep. sanguis). In a similar vein, University of Rochester researchers have published information indicating that cranberry juice may be effective in preventing tooth decay. The research focuses on the inhibiting effect of compounds found in cranberries against a key bacterium blamed for the formation of cavities. They logically conclude that this inhibition parallels the way that cranberries prevent urinary tract infections, i.e., by inhibiting the adherence of pathogens to receptor sites on the surface of the bladder.

Cranberry juice contains substantial quantities of α-D-mannopyranoside, the cyclic ring form of D-mannose that exists in an aqueous medium. Consistent with the teaching of my above-referenced patent, the cis-placement of the two adjacent hydroxyl groups on the mannopyranoside ring apparently facilitates their attachment to the receptive sites on the protein pellicle, to which the lectins from the Staph. species would normally attach. It is also projected that, similar to the effective functionality of the mannan and galactomannan polysaccharides, as disclosed in U.S. Pat. No. 6,126,961, such related polymers, and shorter versions thereof (e.g., mannose and/or galactose dimers, trimers and various oligomers), could also function in that manner. In some cases, the polysaccharides may have side chains only, which contain such cis-oriented hydroxyl groups. Examples of such polysaccharides, where either the main chain and/or the side chains contain cis-oriented hydroxyl groups, are guar gum and xanthan gums. The full content of U.S. Pat. No. 6,126,961 is incorporated herein by reference.

Supportive of this approach is information provided in a 1998 publication in the Journal of the American Dental Association, in which it was reported that a unique cranberry juice component, a high-molecular-weight nondialysable material (NDM), has the ability to reverse and inhibit the coaggregation of certain oral bacteria responsible for dental plaque and periodontal disease in vitro. Further, a 2002 paper in Critical Reviews in Food Science and Nutrition reported on a preliminary clinical trial using a mouthwash containing cranberry NDM. Saliva samples of the experimental group showed a two order of magnitude reduction in the cavity forming Streptococcus mutans bacterial colonies compared with the placebo group. To the inventor's knowledge, no information has been published regarding the identify of the nondialysable material, but it is conjectured that it comprises similar cis-oriented sugars that have been found to adsorb intestinal pathogens.

Accordingly, the present invention results from an attempt to extend the application of these sugar-based bacterially-adsorptive materials to products serving the oral care field, in order to reduce the population of undesired microbial species in the oral cavity. Included in the category of undesirable microorganisms are those responsible for plaque formation, as well as those associated with a variety of oral diseases, such as gingivitis and periodontitis.

### SUMMARY OF THE INVENTION

It is, therefore, an object of the present invention to provide an oral care product and related method that improves upon the state of the art associated with reduction of undesirable oral flora associated with a range of oral afflictions and diseases. Such afflictions and diseases include dental plaque, caries, gingivitis, periodontitis and all such other oral conditions in which unhealthy bacteria and fungi play a causative role.

It is a further object of the present invention to provide an oral care product and related method for determining if the techniques acquired in reducing intestinal populations of bacterial pathogens can be adapted to the upper end of the alimentary canal, specifically the oral cavity, so as to reduce the impact of bacteria and fungi in that area on surrounding oral tissue.

It is, yet, a further object of the present invention to provide an oral care product and related method that involves identifying which saccharides can serve as bacterial and fungal adsorbents for unwanted microorganisms, and to then determine the polymer size and conformation that best function in that capacity.

It is, still, a further object of the present invention to provide an oral care product and related method for determining which oral care products can make use of these teachings and to identify the nature, level and manner of incorporation of the saccharide materials into such oral care products.

The foregoing and related objects are accomplished by an oral care product that reduces the level of undesirable microorganisms, which includes mono- and polymeric saccharides, in which the saccharides comprise cis-hydroxy sugars, e.g., mannose, galactose, galactosamine, fucose, arabinose, rhamnose and combinations thereof. The foregoing sugars may be in either, or both, monomeric and polymeric form in a single oral care product. The oral care product may be, for example, a dentifrice, mouthwash, chewing gum or lozenges.

It has been discovered that the incorporation of so-called cis-hydroxy sugars, particularly in polymers that include them, into oral care formulations will result in lower incidences of dental caries. Cis-hydroxy sugars will also beneficially impact oral diseases, since such materials will attract and effectively neutralize the untoward effects of oral pathogens and other unwanted oral flora. This is a result of the sugar species' attachment to receptor sites on hard-(tooth) and soft-(mucosal) surfaces to which caries-causing bacteria, and related unwanted species, tend to attach. The greater the degree of sugar attachment, from such cis-sugars present as monosaccharides and/or components of poly- and oligo-saccharides (all hereinafter referred to as “saccharides”), the lesser the ability for their corresponding sugar structures on bacterial surfaces to attach to such sites. In general increasing concentrations of such saccharide levels in the oral careformulation (e.g., a dentifrice) correspond to increasing degrees of bacterial attachment. The identity of the various cis-sugars is readily available in U.S. Pat. No. 6,126,961, referenced above. A preferred application of the present invention is the use in dentifrice compositions, although other products that contact the oral cavity, such as mouth rinses, chewing gum, lozenges, and even dental floss, will also benefit from this invention.

Definitions—For the purpose of this application, the follow definitions will apply:

“Isomer”—Any of two or more molecular substances that are composed of the same elements in the same proportions but that differ in properties owing to differences in the arrangement of atoms.

“Stereoisomerism”—This is the arrangement of atoms in molecules whose connectivity remains the same but their arrangement in space is different in each isomer.

“Geometric isomerism”—In this application this term is considered synonymous with stereoisomerism. Stereoisomers and geometric isomers, respectively, are those molecules which are described by these terms. Both types of isomerism, for these purposes, describe the orientation of functional groups at the ends of a bond around which no rotation is possible. There are two forms of a geometric isomer, the cis- and trans-versions. The form in which the substituent groups are on the same side of the bond, that doesn't allow rotation, is called cis; the form in which the substituents are on opposite sides of the bond is called trans.

“Diastereomer”—In the present context, is an alternate term for both stereoisomer and geometric isomer.

“Epimer”—Refers to a molecule differing from a related isomer only in the spatial arrangement around a single carbon atom.

“Saccharide”—A category that includes simple monomeric sugars as well as macromolecular substances comprised of a number of these monosaccharide groups and derivatives thereof.

“Dental plaque”—Refers to the complex mass of bacteria that forms on the teeth, and is the etiological factor responsible for two of the most prevalent human diseases, caries and periodontal disease.

“Dental caries”—Refers to acid-induced changes in tooth structure caused by relatively specific acid-generating bacterial populations inhabiting the teeth in dental plaque.

“Periodontal disease”—While the causative microorganisms responsible for diseases of the gums surrounding the teeth (i.e., gingivitis and periodontitis) are not clearly defined, the role of plaque bacteria in their etiologies has been proven definitively.

Other objects and features of the present invention will become apparent when considered in view of the following detailed description of the invention, which provides certain preferred embodiments and examples of the present invention.

It should, however, be noted that the accompanying detailed description is intended to discuss and explain only certain embodiments of the claimed invention and is not intended as a means for defining the limits and scope of the invention.

### DETAILED DESCRIPTION OF THE PREFERRED EMBODIMENTS

As taught in U.S. Pat. No. 6,126,961, the entirety of which is incorporated herein by reference, there are certain common structural features in some simple sugars and more complex materials, primarily polysaccharides that contain such sugars, which predispose these compounds to bind to bacterial pathogens and/or tissue receptor sites. Such binding can impede the ability of bacteria, relative to conforming surface structures, to otherwise attach to tissues and propagate on site. Without attachment the bacteria are more readily removed from the area by normal body processes. The simple sugars that possess that common feature were identified as galactose, fucose, mannose, rhamnose, and arabinose, and derivatives thereof, such as galactosamine, lactose, as well as a variety of longer chain homo- and complex polysaccharides that comprise these sugars. Included in the latter are a number of mannans and galactomannans, which are characteristic of a variety of naturally-occurring gums that are found in commercial food and cosmetic products. Polymers of mannose and galactomannose are also found in the cell walls of yeasts, which are often present in human and animal foods. Among the more common gums used in food which include these saccharides are xanthan, konjac, guar gum, locust bean gum, and carrageenan.

I have concluded that the same ability manifested by these saccharides, either in the mono-form (e.g., mannose) or in complex form (e.g., guar gum), to reduce bacterial populations in the intestinal tract is also applicable to the initial portion of the gastrointestinal tract, i.e., the oral cavity. For example, these so-called “cis-” structured saccharides can be incorporated into various dentifrice compositions. More specifically, the sugars, in their monomeric and/or their complex polymer forms, can be included in the humectant and/or the thickening (gum) components of the dentifrices, since virtually all dentifrices contain these materials at significant levels. With respect to humectancy, sugars, by virtue of their poly-hydroxy structures, are themselves humectants by nature. They are readily compatible with commonly-used humectants, such as sorbitol, glycerin and various polyethylene glycols. The level of use of these saccharides in the inventive dentifrice compositions can range from about 2% to about 25% of the dentifrice.

In addition some of the thickeners incorporated into dentifrices can be also replaced, or augmented with polysaccharide gums thickeners, and more preferably partially-hydrolyzed versions thereof, and which are also cis-hydroxy bearing sugar structures. Since it is important to have adhesion to the receptor site by smaller molecules, because larger ones may have competing solubilizing tendencies, in general the monosaccharides and shorter-chain combinations of them are preferred to the common thickening gums that may contain the cis-hydroxy moieties. The list of thickeners that have been used in major dentifrice U.S. formulations includes silica aerogels, pyrogenic silica, silica precipitates, carboxymethyl cellulose, carboxyvinyl polymers, carrageenan, and xanthan gum. The latter contains a side chain that includes, in part, a mannopyranose structure, but this is less effective, and therefore less preferred than a thickener such as konjac gum, which is a more linear (mannose)-(acetylated glucose) polysaccharide. Guar gum is another example of a preferred thickener that would provide receptor-site blocking, since it consists of a (1-4) linked β-D mannose chain with a side chain of (1-6) linked α-D galactose. Other useful gums are tara bean gum and locust bean gum, the structure of which is depicted below, to show the nature of cis-hydroxy structured polysaccharides. 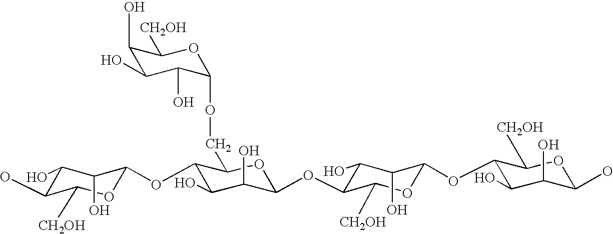
Locust bean gum, a β-(1,4)-D-mannose structure

The level of use of these cis-hydroxy containing polysaccharides, including partial hydrolysis products thereof, may range from about 2% of the dentifrice to about 25% thereof. One such partially-hydrolyzed gum is Novartis' Benefiber™, which has an average molecular weight of about 20,000, and is obtained as a water-soluble dietary fiber by digestion of guar gum with beta-D-endomannanase. Guar gum, generally, has a MW of about 200,000. The water solubility facilitates its inclusion in dentifrice compositions, as well as other oral care formulations hereinafter discussed. Other ingredients in the dentifrice compositions are familiar to those skilled in the art of their fabrication. The smooth cohesive paste compositions can readily accommodate the inventive materials, and the ordinary components need not be altered substantially in their nature except as indicated above, i.e., the abrasive, the humectant, the surfactant, the binder, the flavor, and any active agent needed to provide therapeutic or other special activity.

When the intent is to include the inventive materials in oral rinse formulations, there is significant leeway in material selection in their formulation. Virtually all mouthwashes are formulated with antimicrobial agents; even those directed primarily at the control of oral malodor which contain materials that impact odor-causing bacteria as well as the volatile sulfurous malodorants. The list of antimicrobial agents in mouthwashes is large, including such agents as chlorhexidine, triclosan, essential oils, cetyl pyridinium chloride, and chlorine dioxide. In addition to some antimicrobial agent, mouthwashes also generally contain alcohol, a flavor, a color, a humectant, a surfactant, and water. The humectant usually is glycerin, which adds “body” to the product, as well as inhibiting crystallization of salts around the closure.

The compositions of the present invention are intrinsically compatible with all of these mouthwash types, and when the longer chain cis-saccharides are used, the latter can also contribute to the “body” or mouthfeel of the composition. For example guar gum, gum arabic, konjac and locust bean are used as food thickeners. The use of these materials will reduce the level needed for a humectant to impart mouthfeel, since significantly lower quantities of thickening gums provide a similar effect as higher levels of glycerin and sorbitol humectants. The use level of the inventive materials will therefore depend on the total overall amount of the shorter and longer chain cis-saccharides deemed appropriate for oral bacteria reduction, and a balance of the shorter (e.g., monosaccharides), partially hydrolyzed guar gum [as cf. Benefiber]) and the longer chain gums mentioned above.

The total quantity of cis-saccharides (short and longer chain) found useful in mouthwashes to provide beneficial reduction of undesired oral flora is from about 2% to about 15% of the total mouthwash composition. The undesired flora include those bacteria generally associated with plaque formation and gum diseases, which organisms cover the range from aerobic, facultative aerobic, facultative anaerobic, to the anaerobic bacteria. The category of undesired microorganisms also includes the putrefactive organisms, generally the anaerobes and facultative anaerobes, which are associated with the degradation of proteinaceous cellular debris and other organic matter in the oral cavity, to form the volatile sulfur compounds (VSCs) responsible for non-systemic oral malodor. The following tabulation lists the various bacteria that are frequently encountered in plaque, dental caries, gingivitis and periodontitis:

| | | Dental | | |
|---|---|---|---|---|
| BACTERIUM | Plaque | caries | Gingivitis | Periodontitis |
| Streptococcus sanguis | ++ | ++ | ++ | + |
| Streptococcus mutans | ++ | ++ | 0 | 0 |
| Actinomyces viscosis | + | + | ++ | + |
| A. israelii | + | + | ++ | ++ |
| Lactobacillus sp. | + | + | 0 | 0 |
| Propionibacterium | 0 | + | + | ++ |
| acnes | | | | |
| Bacteroides sp. | 0 | 0 | + | ++ |
| Selenomonas sputagena | 0 | 0 | + | ++ |
| Large spirochetes | 0 | 0 | 0 | ++ |

++ = Frequently encountered in high proportions;
+ = Frequently encountered in low to moderate proportions;
0 = Sometimes encountered in low proportions or not detectable. Modified from Davis, et al.: Microbiology. 4th ed. J. B. Lippincott. Philadelphia, 1990. In addition to the organisms shown on the chart, oral problems can be caused by Porphyromonas and Fusobacterium sp. E.g., Porphyromonas gingivalis and Porphyromonas endodontalis can be associated with periodontitis or periapical infections.

With regard to the above-mentioned range of about 2% to about 15% of total cis-saccharides found useful in mouthwashes with these inventive compositions, there is an upper limitation on the amount of the longer-chain cis-saccharides that may be used because these are generally food gums that are used to increase the viscosity of (i.e., thicken) aqueous systems. When standard commercial food thickeners, such as guar gum, konjac and locust bean gum, comprise a portion of the total cis-saccharide components in the mouthwash, their level of use can be from about 0.1% up to about 1.5% of the total product. Partially hydrolyzed cis-saccharides, depending upon their degree of hydrolysis, may represent up to about 10% of the composition. Smaller cis-saccharides, such as the monomer sugar and short-chain products with average molecular weights of up to about 1,000, may be employed at levels of up to about 15%, with the limitation that the total quantity of cis-saccharides does not exceed about 15% of the total composition. The optimum level of use will depend upon a number of factors, which include a correlation of undesirable flora reduction (as determined by salivary microbiology studies), mouthfeel, taste, stability, and such other parameters familiar to those with skills in the art of oral product formulation.

To include the inventive materials in chewing gum formulations, there is a relatively simple approach that can be followed in order to provide compositions with effective levels of the cis-saccharides. Chewing gum basically consists of a tasteless gum base, sweeteners, flavors, active ingredients and functional ingredients, including texture-regulating agents and colors. Bulk sweeteners represent about 60% of the product, gum base about 30%, flavors 3-4%, functional ingredients about 3%, softeners about 2% and high intensity sweeteners (e.g., aspartame) generally about 0.5%. The most commonly used bulk sweeteners for sugar products are sucrose and dextrose. For sugar-free chewing gum, polyols: sorbitol, xylitol, isomalt and maltitol are mainly employed. The gum base is the part that is not dissolved during chewing. Its composition of resins, elastomers, fillers, waxes, fats and emulsifiers determines the texture, flavor release and taste longevity of the final gum product.

A particular advantage of using chewing gums to deliver the cis-saccharides of this invention lies in the efficacy of their delivery from this substrate. When the active ingredients are released from the gum, they are efficiently distributed throughout the mouth during the process of extended chewing. The sustained release of such ingredients from chewing gum means that their oral residence time is much longer than when released using other oral delivery methods such as dentifrices and mouthwashes. This longer residence time is likely to prolong the effect of the adsorbent saccharides on oral flora. Because of the varying water-solubilities of the cis-saccharides, from the high solubility of monomeric mannose to the moderate to low solubility of the partially hydrolyzed gums, such as Benefiber, to the minimal solubility of the non-hydrolyzed good gums, such as konjac or locust bean gum, it is possible to judiciously select the proper blend of fast- and slow-releasing saccharides in order to provide a protracted, and continuous presence of these protective materials in the oral cavity during the extend period of mastication. The cis-saccharides of the inventive composition may represent a significant portion of the typical ˜60% “sweetener” fraction of the chewing gum. Since these saccharides do not possess the typical sweetness of the sucrose and/or corn syrup and/or dextrose sweeteners which usually comprise this fraction, their partial replacement of the usual sweeteners will simply require a slight increase in the high-intensity sweetener portion, such as with aspartame or acesulfame-K.

In general, the inventive saccharides may represent from about 5% to about 35% of the “sweetener” fraction of the gum, corresponding to from about 3% to about 21% of the finished gum. Of the total sweetener fraction, mannose per se can represent from about 0% up to about 10% of that fraction, partially-hydrolyzed gums from about 0% up to about 20% of that fraction, and food gums from about 0% up to about 15%. The latter usage depends, to a large degree, on the nature and solubility of the particular food gum, since the many qualifying cis-saccharide varieties have a range of properties which will determine their release from the finished gum stick as a function of their differential solubility in saliva and the humectant component of the finished gum.

The commercial production of the inventive gum composition is fairly straight-forward, and is basically comprised of the following steps: The gum base, whether natural or synthetic, is melted at a temperature of about 240° F., until it has the viscosity of thick maple syrup, and filtered through a fine mesh screen. The clear base, while still hot and melted, is then put into a mixing vat, where the temperature is lowered to about 200° F. and the other ingredients are then added. These include: the humectants (the standard sugars including the selected blend of cis-saccharides or, for sugar-free gums, such polyols as sorbitol, xylitol, isomalt and maltitol), the flavors, the softeners (e.g., glycerin, lecithin, maltitol, sorbitol), the high intensity sweeteners, and such other desired active ingredients. The latter can be materials that typically contribute to dental health, tooth whitening and fresh breath. For example, carbamide may be used to neutralize plaque acids in order to prevent caries, baking soda or calcium carbonate may be used for its well known teeth-whitening property, and zinc acetate or green tea may be added for breath-freshening purposes. The homogenized mixture is then poured onto cooling belts, and cooled with cold air. The product is then subjected to mechanical shaping operations, such as extrusion, rolling and cutting. The chunks of gum are then put aside to set for 24 to 48 hours before final processing into the desired finished shape.

The following Examples illustrate the inventive concept in accordance with, and as disclosed herein, the use of simple and polymeric cis-OH sugar materials in oral care compositions, in order to bind to, and reduce the presence of, undesired microorganisms in the oral cavity. It is to be understood that these Examples are provided by way of illustration only, and nothing therein should be taken as a limitation upon the overall scope of the invention.

### EXAMPLE 1

This Example illustrates the use of mannose, in monomeric form, in fabricating a dentifrice that suppresses the tendency of the user to develop dental plaque which can lead to both dental caries of gum diseases:

| Component | Level in Dentifrice |
|---|---|
| Glycerin | 25.00% |
| Dicalcium phosphate dihydrate | 37.00% |
| Water | 25.38% |
| Mannose powder | 5.00% |
| Mint flavor | 0.05% |
| Carrageenan | 0.90% |
| Locust-bean gum, partially | 5.00% |
| hydrolyzed (Benefiber) | |
| Sodium monofluorophosphate | 0.75% |
| Sodium benzoate | 0.50% |
| Sodium saccharin | 0.20% |
| Poloxamer 407 | 0.20% |
| FD&C Green #3 (0.01% aqueous) | 0.02% |

Preparation:

Disperse the mannose, Benefiber™, saccharin, benzoate, monofluorophos-phate, and carrageenan into the premixed glycerin and Poloxamer™. Heat to between 40° and 60° C. and maintain at this temperature for about 15 to 20 minutes while milling the mixture to a paste. Add the water containing the predissolved color, and mill all components thoroughly at the same temperature; then cool the vessel to about 40° C. before adding the dicalciun phosphate. Homogenize the mixture by milling, and then vacuumize the vessel to remove air bubbles. Next add the flavor and mix in at a low speed, avoiding the entrapment of air, until a homogeneous paste is achieved.

### EXAMPLE 2

This Example illustrates the use of both konjac and guar gums to formulate a low-water dentifrice which is effective in both abrading the pellicle on teeth to which plaque attaches, and also removing plaque-forming organisms, such as Strep. mutans and Strep. sanguis, to minimize the subsequent development of both dental caries of gum diseases. A combination of hydrated silica abrasives is used for physical abrasion, and the man-nose-containing food gums provide the adsorptive means for attachment and removal of the undesired plaque formers once the dentifrice is dispersed in saliva during brushing:

| Component | Level in Dentifrice |
|---|---|
| Sorbitol (70% aqueous) | 55.960% |
| Glycerin | 15.000% |
| NaH₂PO₄•H₂O | 0.050% |
| Na₂HPO₄•2H₂O | 0.200% |
| Sodium saccharin | 0.250% |
| Syloid 63 | 3.00% |
| Syloid 74 | 13.00% |
| Flavor (spearmint) | 0.920% |
| Water | 0.061% |
| Sodium fluoride | 0.243% |
| Carbopol 940 | 0.250% |
| Konjac gum | 3.200% |
| Guar gum | 3.200% |
| Sodium lauryl sulfate (28.8% aqueous) | 4.000% |
| Green color (1% aqueous) | 0.666% |

The preparation of the dentifrice is straightforward, in a manner familiar to those with experience in the art of tooth paste production. It involves the initial dispersal of the gums, Carbopol, saccharine and salts in the sorbitol/glycerin combination, followed by thorough mixing. Thereafter the Syloids, flavor, water and sodium lauryl sulfate are added, and the mixture then milled until a smooth paste is achieved. As a final step the aqueous green color is added to the mixing composition, which is then further blended until the color is uniformly distributed.

### EXAMPLE 3

This Example illustrates the use of a water-soluble partially-hydrolyzed guar gum in the formulation of a fluoride-containing oral rinse, which will suppress the tendency of a regular user to develop dental plaque, a precursor to both dental caries of gum diseases:

| | % by |
|---|---|
| Component | weight |
| Ethyl alcohol | 5.00 |
| Mint flavor (spearmint oil 45%, peppermint oil 10%, | 0.25 |
| methyl salicylate 25%, menthol 20%) | |
| Glycerin | 15.00 |
| Partially-hydrolyzed guar gum, MW 20-30,000 | 8.00 |
| [Sunfiber, from Taiyo Kagaku Co., Ltd. Japan] | |
| Polyoxyethylene/poloxypropylene block polymer; | 1.00 |
| (Poloxamer 338) | |
| Saccharin, sodium | 0.05 |
| Sodium fluoride | 0.05 |
| Sodium benzoate | 0.10 |
| FD&C dyes to desired color | q.s. |
| Water (deionized) | q.s. |
| pH adjusted to 5.5-6.0 | |

The product is prepared by combining the alcohol, Poloxamer™ and glycerin, followed by addition of the flavor with mixing. The SUNFIBER™ is dispersed in the liquid, followed by addition of the saccharin, fluoride and benzoate, for subsequent dispersal. Thereafter water is added to the mixture to ˜99% of the required volume, and the dye mixture then stirred in. At that point, the pH is measured and the solution adjusted, if necessary, to the specified range with either 10N NaOH or conc. HCl, as needed. The final quantity of water is added, and the mixture is stirred to uniformity.

### EXAMPLE 4

This Example illustrates the use of a combination of mannose and guar gum, a longer-chain cis-saccharide, in the formulation of a mouthwash with an enhanced mouth-feel, which will last longer in the mouth than a less viscous rinse, and will provide enhanced protection against plaque formation and potential gum diseases:

| | % by |
|---|---|
| Component | weight |
| Ethyl alcohol | 5.00 |
| Cherry flavor | 2.40 |
| Glycerin | 10.00 |
| Mannose | 5.00 |
| Guar gum | 0.50 |
| Polyoxyethylene/poloxypropylene | 2.00 |
| block polymer; (Poloxamer 338) | |
| Saccharin, sodium | 0.05 |
| FD&C Red #40 (1% solution) | 2.40 |
| Sodium benzoate | 0.10 |
| Water (deionized) | q.s. |
| pH adjusted to 5.5-6.0 | |

The product is prepared by combining the alcohol, POLOXAMER™ and glycerin, followed by addition of the flavor with mixing. The guar gum and mannose are sequentially dispersed in the liquid, followed by addition of the saccharin and benzoate, for subsequent dispersal. Thereafter water is added to the mixture to ˜99% of the required volume, and the dye solution is then stirred in. At that point, the pH is measured and the solution adjusted, if necessary, to the specified range with either 10N NaOH or conc. HCl, as needed. The final quantity of water is added, and the mixture is stirred to uniformity.

### EXAMPLE 5

This Example illustrates the use of a mixture of mannose and a partially hydrolyzed cis-saccharide in the formulation of a chewing gum which will provide protection against plaque formation and potential gum diseases. The composition of the chewing gum is as follows:

| Bulk sweetener: | |
|---|---|
| Beet sugar | 24%, |
| Corn syrup | 24% |
| Latex Gum base: | 30% |
| Flavors (mint extract): | 3.5% |
| Cis-saccharide component: | |
| Mannose | 5.0% |
| Benefiber ™ | 10.0% |
| Glycerin: | 2.0% |
| Calcium carbonate powder | 1.0% |
| Acesulfame-K | 0.5% |

The commercial production of the inventive gum composition proceeds as follows: The gum base is heated to about 240° F., until readily flowable. It is then filtered through a fine mesh screen, and the clarified, hot base is then put into a mixing vat, where the temperature is lowered to about 200° F. Thereafter the other ingredients are then added sequentially; first the sugar then the corn syrup, then the glycerin, followed by the mannose and Benefiber powders. While still at 200° F., the calcium carbonate powder and acesulfame are mixed into the thick liquid, and finally the liquid is reduced in temperature where it is still fluid enough for the mint extract to be uniformly mixed into the mixture. The homogenized mixture is then poured onto cooling belts, and cooled with cold air. The mass is then rolled and cooled, for a minimum of 24 hours, before being cutting into stick form, and packaged.

### EXAMPLE 6

This Example illustrates the use of a mixture of a partially hydrolyzed cis-saccharide and a longer-chain cis-saccharide in the formulation of a sugar-free chewing gum that will provide protection against plaque formation and potential gum diseases.

The composition is as follows:

| Bulk sweetener: | |
|---|---|
| Mannitol | 24%, |
| Xylitol | 24% |
| Latex Gum base: | 30% |
| Flavors (mint extract): | 3.5% |
| Cis-saccharide component: | |
| Guar Gum | 5.0% |
| Benefiber ™ | 10.0% |
| Glycerin: | 2.0% |
| Calcium carbonate powder | 1.0% |
| Acesulfame-K | 0.5% |

The commercial production of the inventive gum composition proceeds as follows: The gum base is heated to about 240° F., until readily flowable. It is then filtered through a fine mesh screen, and the clarified, hot base is then put into a mixing vat, where the temperature is lowered to about 200° F. Thereafter the other ingredients are then added sequentially; first the mannitol and the xylitol, then the glycerin, followed by the guar gum and Benefiber powders. While still at 200° F., the calcium carbonate powder and Acesulfame are mixed into the thick liquid, and finally the liquid is reduced in temperature where it is still fluid enough for the mint extract to be uniformly mixed into the mixture. The homogenized mixture is then poured onto cooling belts, and cooled with cold air. The mass is then rolled and cooled, for a minimum of 24 hours, before being cutting into stick form, and packaged.

While only several embodiments of the present invention have been shown and described, it will be obvious to those skilled in the art that many modifications may be made to the present invention without departing from the spirit and scope thereof

What is claimed is:

1. An oral care product for reducing undesirable microorganisms in the oral cavity, comprising:
an oral care composition including a member selected from the group consisting of a monomeric saccharide having at least one cis-hydroxy sugar, a polymeric saccharide having at least one cis-hydroxy sugar, and a combination thereof.

2. The oral care product for reducing undesirable microorganisms in the oral cavity according to claim 1, wherein said oral care product is a member selected from the group consisting of a dentifrice, a mouthwash, a chewing gum and a lozenge.

3. The oral care product for reducing undesirable microorganisms in the oral cavity according to claim 1, wherein said at least one cis-hydroxy sugar of said monomeric saccharide is a member selected from the group consisting of mannose, galactose, galactosamine, fucose, arabinose, rhamnose, and a combination thereof.

4. The oral care product for reducing undesirable microorganisms in the oral cavity according to claim 1, wherein said at least one cis-hydroxy sugar of said polymeric saccharide is a member selected from the group consisting of mannose, galactose, galactosamine, fucose, arabinose, rhamnose, a food gum and a combination thereof.

5. The oral care product for reducing undesirable microorganism in the oral cavity according to claim 4, wherein said food gum is a member selected from the group consisting of guar gum, xanthan gum, pectin, gum tragacanth, gum arabic, locust bean gum, algin, carrageenan, and a combination thereof.

6. The oral care product for reducing undesirable microorganisms in the oral cavity according to claim 5, wherein said food gum includes a partially-hydrolyzed composition thereof.

7. The oral care product for reducing undesirable microorganism in the oral cavity according to claim 1, wherein said oral care product is a dentifrice and said monomeric saccharide and said polymeric saccharide have a total combined weight of approximately 2% to approximately 25%, by weight, of said dentifrice.

8. The oral care product for reducing undesirable microorganism in the oral cavity according to claim 1, wherein said oral care product is a mouthwash and said monomeric saccharide and said polymeric saccharide have a total combined weight of approximately 2% to approximately 15%, by weight, of said mouthwash.

9. The oral care product for reducing undesirable microorganisms in the oral cavity according to claim 8, wherein said monomeric saccharide, in combination with short-chain saccharides having average molecular weights of up to approximately 1,000, constitute up to approximately 15%, by weight, of said oral care product.

10. The oral care product for reducing undesirable microorganisms in the oral cavity according to claim 9, wherein said short-chain saccharides constitute up to approximately 10%, by weight, of said oral care product.

11. The oral care product for reducing undesirable microorganisms in the oral cavity according to claim 8, wherein said polymeric saccharide constitutes from approximately 0.1% to approximately 1.5%, by weight, of said oral care product.

12. The oral care product for reducing undesirable microorganism in the oral cavity according to claim 1, wherein said oral care product is a chewing gum and said monomeric saccharide and said polymeric saccharide have a total combined weight of approximately 3% to approximately 21%, by weight, of said chewing gum.

13. A method for reducing undesirable microorganisms in the oral cavity, comprising the steps of:
incorporating a member selected from the group consisting of a monomeric saccharide having at least one cis-hydroxy sugar, a polymeric saccharide having at least one cis-hydroxy sugar, and a combination thereof into an oral care composition for making an oral care product; and,
placing said oral care product into the oral cavity of a person or non-human animal.

14. The method for reducing undesirable microorganisms in the oral cavity according to claim 13, wherein said oral care product is a member selected from the group consisting of a dentifrice, a mouthwash, a chewing gum and a lozenge.

15. The method for reducing undesirable microorganisms in the oral cavity according to claim 13, wherein said at least one cis-hydroxy sugar of said monomeric saccharide is a member selected from the group consisting of mannose, galactose, galactosamine, fucose, arabinose, rhamnose, and a combination thereof.

16. The method for reducing undesirable microorganisms in the oral cavity according to claim 13, wherein said at least one cis-hydroxy sugar of said polymeric saccharide is a member selected from the group consisting of mannose, galactose, galactosamine, fucose, arabinose, rhamnose, a food gum and a combination thereof.

17. The method for reducing undesirable microorganisms in the oral cavity according to claim 16, wherein said food gum is a member selected from the group consisting of guar gum, xanthan gum, pectin, gum tragacanth, gum arabic, locust bean gum, algin, carrageenan, and a combination thereof.

18. The method for reducing undesirable microorganisms in the oral cavity according to claim 17, wherein said food gum includes a partially-hydrolyzed composition thereof.
